(19) United States
(12) Patent Application Publication
(10) Pub. No.: US 2019/0195848 A1
(43) Pub. Date: Jun. 27, 2019

(54) VOC DETECTING AND WARNING METHOD

(71) Applicant: Microjet Technology Co., Ltd., Hsinchu (TW)

(72) Inventors: Hao-Jan Mou, Hsinchu (TW); Ta-Wei Hsueh, Hsinchu (TW); Shih-Chang Chen, Hsinchu (TW); Chi-Feng Huang, Hsinchu (TW); Yung-Lung Han, Hsinchu (TW); Hsuan-Kai Chen, Hsinchu (TW)

(73) Assignee: Microjet Technology Co., Ltd., Hsinchu (TW)

(21) Appl. No.: 16/191,792

(22) Filed: Nov. 15, 2018

(30) Foreign Application Priority Data
Dec. 25, 2017 (TW) 106145552

(51) Int. Cl. G01N 33/00

(52) U.S. Cl. CPC G01N 33/0047; G01N 33/0063; G01N 33/0073

(57) ABSTRACT

A VOC detecting and warning method is provided. Firstly, an actuating-and-sensing module having a gas transportation actuator and a gas sensor is provided. Then, the gas transportation actuator is enabled to guide a specific amount of gas to the gas sensor in each monitoring time interval, so that each monitored value of a VOC in the specific amount of the gas corresponding to each monitoring time interval is acquired. A plurality of the monitoring time intervals define a time unit. Then, the monitored values during the time unit are added up to obtain a metabolism comparison value. If the metabolism comparison value is larger than a warning threshold value that is defined according to an upper limit of the VOC that a human subject inhales per time unit, the actuating-and-sensing module issues an emergency call, thereby providing a user with a protective measure.

### FIELD OF THE INVENTION

The present disclosure relates to a VOC detecting and warning method, and more particularly to a VOC detecting and warning method, which utilizes an actuating-and-sensing module to guide gas and monitor volatile organic compounds of the gas.

### BACKGROUND OF THE INVENTION

Recently, the air pollution problem becomes more and more serious. Consequently, people pay much attention to the methods of avoiding the harmfulness of various pollutants in the air. As known, volatile organic compounds (VOCs) are the common pollutants that are present in the air. The VOCs are in a gaseous form at room temperature and have strong volatility. For example, the VOCs include formaldehyde, toluene, xylene, ethylbenzene, propylene benzene, and so on. In addition to the exhaust gases from fuel combustion and vehicle transportation, the sources of the VOCs are diverse. For example, the VOCs are often dispersed in indoor environments from construction and decorative materials such as paints, coatings and adhesives. The VOCs may cause damage to the human bodies over the years. If the concentration of the VOCs in a room exceeds a specific concentration, people feel headache, nausea, vomit and limb weakness in a short time. In severe cases, people may have convulsions, coma and memory loss. Moreover, the VOCs harm people's livers, kidneys, brains and nervous systems, and they also contain many carcinogens.

However, the current technologies of detecting the VOCs still have some drawbacks. For example, in case that the volume of the detection device is small, the detection device is usually unable to immediately and accurately detect the VOCs. In addition, there is no detection device on the present market that can be carried and warn the user in real time when the amount of the VOCs in the environment reaching a hazard level is detected.

Therefore, there is a need of an improved VOC detecting and warning method in order to overcome the drawbacks of the conventional technologies that the accuracy of detecting the VOCs is low and fails to warn the user in real time.

### SUMMARY OF THE INVENTION

An object of the present disclosure provides a VOC detecting and warning method, which utilizes a gas transportation actuator of an actuating-and-sensing module to guide the gas to a gas sensor of the actuating-and-sensing module. A plurality of monitoring time intervals define a time unit. Then, the gas sensor monitors the VOCs during the time unit and generates each monitored value corresponding to each monitoring time interval of the time unit. The monitored values are added up to obtain a metabolism comparison value. Then, the metabolism comparison value is compared with a warning threshold value. The warning threshold value defines a critical amount that a human subject inhales per time unit. For example, a value that exceeds the warning threshold value may indicate that the concentration of the VOCs has adverse effects on the human subject. If the metabolism comparison value is larger than the warning threshold value, the actuating-and-sensing module issues an emergency call to warn the user. Since the gas is guided to the gas sensor, the accuracy of the VOC detection is increased. In addition, when the amount of VOCs in the environment reaching a hazard level is determined, the emergency call is issued to warn the user in real time. Therefore, the drawbacks of the conventional technologies that the accuracy of detecting the VOCs is low and fails to warn the user in real time can be addressed.

In accordance with an aspect of the present disclosure, a VOC detecting and warning method is provided. Firstly, an actuating-and-sensing module is provided. The actuating-and-sensing module includes a gas transportation actuator and a gas sensor. Then, a gas-guiding and monitoring operation is performed. The gas transportation actuator is enabled to guide a specific amount of gas to the gas sensor, and the gas sensor monitors a VOC of the gas to obtain each monitored value during each monitoring time interval. Then, a calculating operation is performed. A plurality of the monitoring time intervals define a time unit. The monitored values in the time unit are added up to obtain a metabolism comparison value. Finally, a comparing and warning operation is performed. The metabolism comparison value is compared with a warning threshold value. The warning threshold value defines a critical amount that a human subject inhales per time unit. For example, a value that exceeds the warning threshold value may indicate that the concentration of the VOCs has adverse effects on the human subject. If the metabolism comparison value is larger than the warning threshold value, the actuating-and-sensing module issues an emergency call, thereby providing a user with a protective measure.

In an embodiment, the gas-guiding and monitoring operation of the VOC detecting and warning method further includes a step of allowing the actuating-and-sensing module to issue the emergency call if the monitored value is larger than a warning threshold value during the monitoring time interval.

In an embodiment, the actuating-and-sensing module further includes a microprocessor and a transmission module. The microprocessor processes and converts information of the monitored values sensed by the gas sensor into an output data and controls the actuation of the gas transportation actuator. The transmission module transmits the output data, which is processed and converted by the microprocessor, to a connection device, so that the connection device displays, stores and transmits information of the output data.

The above contents of the present disclosure will become more readily apparent to those ordinarily skilled in the art after reviewing the following detailed description and accompanying drawings, in which:

### BRIEF DESCRIPTION OF THE DRAWINGS

FIG. 1 is a flowchart illustrating a VOC detecting and warning method according to an embodiment of the present disclosure;

FIG. 2 is a schematic functional block diagram illustrating an actuating-and-sensing module for implementing the VOC detecting and warning method;

FIG. 3A schematically illustrates a monitoring result of a gas sensor of the actuating-and-sensing module within a specific time unit according to the embodiment of the present disclosure;

FIG. 3B schematically illustrates the relationships between warning threshold values and time units;

FIG. 4 is a schematic perspective view illustrating the actuating-and-sensing module according to the embodiment of the present disclosure; and

FIG. 5 is a schematic cross-sectional view illustrating the actions of the actuating-and-sensing module according to the embodiment of the present disclosure.

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for purpose of illustration and description only. It is not intended to be exhaustive or to be limited to the precise form disclosed.

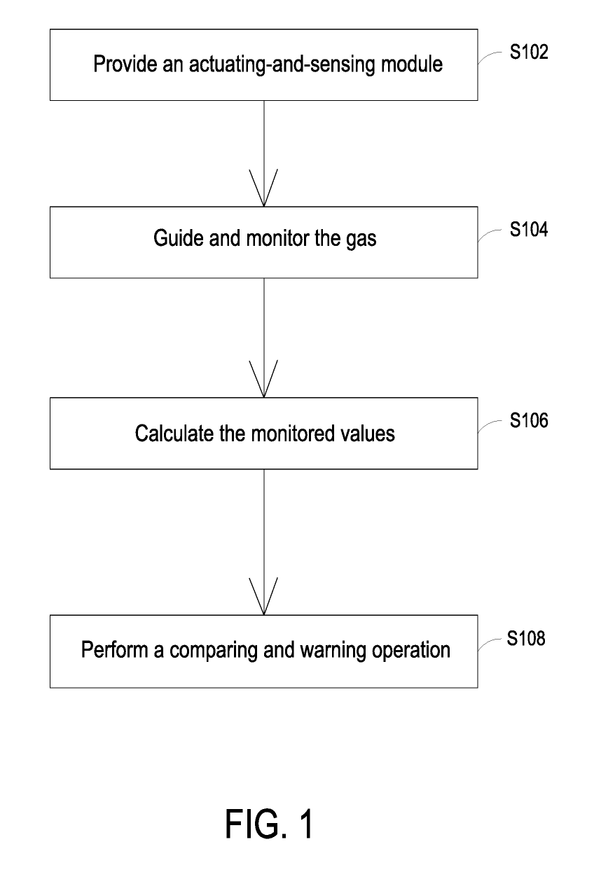

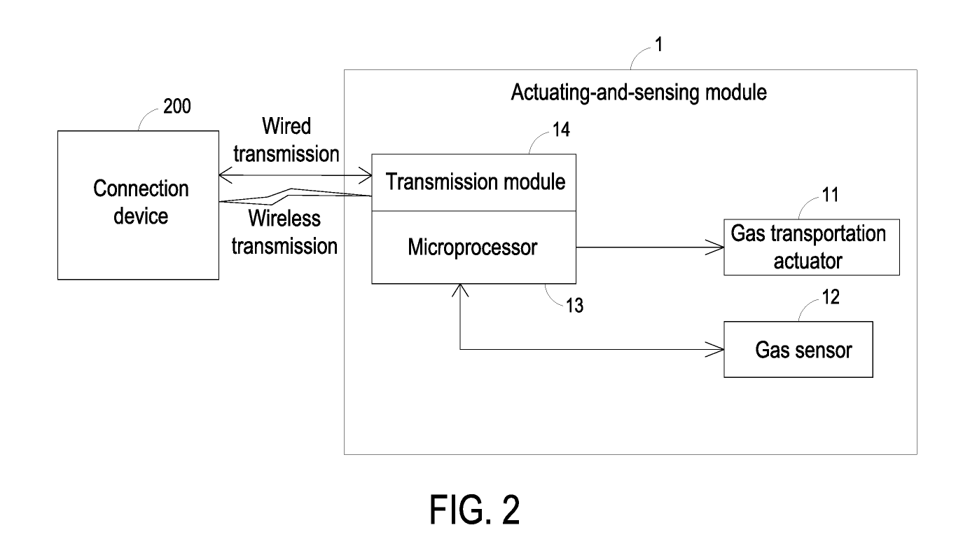

Please refer FIGS. 1 and 2. A VOC detecting and warning method is provided in the present disclosure. The method is implemented by employing at least one actuating-and-transporting module 1, at least one gas sensor 12, at least one gas transportation actuator 11, at least one specific amount of gas, at least one VOC concentration value, at least one monitored value, at least one time unit, at least one metabolism comparison value, at least one warning threshold value, at least one emergency call. The numbers of the actuating-and-transporting module 1, the gas sensor 12, the gas transportation actuator 11, the specific amount of the gas, the VOC concentration value, the monitored value, the time unit, the metabolism comparison value, the warning threshold value, the emergency call are exemplified by one for each respectively in the following embodiments but not limited thereto. It is noted that each of the actuating-and-transporting module 1, the gas sensor 12, the gas transportation actuator 11, the specific amount of the gas, the VOC concentration value, the monitored value, the time unit, the metabolism comparison value, the warning threshold value, the emergency call can also be provided in plural numbers.

Please refer to FIGS. 1 and 2. FIG. 1 is a flowchart illustrating a VOC detecting and warning method according to an embodiment of the present disclosure. FIG. 2 is a schematic functional block diagram illustrating an actuating-and-sensing module for implementing the VOC detecting and warning method. Firstly, in a step S102, an actuating-and-sensing module 1 is provided. As shown in FIG. 2, the actuating-and-sensing module 1 includes a gas transportation actuator 11 and a gas sensor 12. In this embodiment, the actuating-and-sensing module 1 further includes a microprocessor 13 and a transmission module 14, but not limited thereto. When the gas transportation actuator 11 is enabled, a pressure gradient is generated to drive the gas to flow in a specified direction. The hardware structure of the gas transportation actuator 11 will be described later. The gas sensor 12 is used for detecting VOCs of the gas to acquire monitored values of the VOCs. The microprocessor 13 is used for processing and converting the information of the monitored values sensed by the gas sensor 12 into an output data and controlling the actuation of the gas transportation actuator 11. The transmission module 14 transmits the output data, which is processed and converted by the microprocessor 13, to a connection device 200, so that the connection device 200 can display, store and transmit the information of the output data.

In an embodiment of the present disclosure, the connection device 200 may be a display device or a portable electronic device with a wired communication module or a wireless communication module. In response to the emergency call, the connection device 200 may perform a protective measure to notify a user. The protective measure is performed by at least one selected from the group consisting of displaying an image, generating a sound effect, generating a light effect and generating a vibration effect. The transmission module 14 may be a wired transmission module or a wireless transmission module. The wired transmission module may be at least one selected form the group consisting of a USB transmission module, a mini-USB transmission module and a micro-USB transmission module. The wireless transmission module may be at least one selected from the group consisting of a Wi-Fi transmission module, a Bluetooth transmission module, a radio frequency identification (RFID) transmission module and a near field communication (NFC) transmission module. The connection device 200 and the transmission module 14 are not limited to the above embodiments and may be varied according to practical requirements.

Please refer to FIG. 1. In a step S104, a gas-guiding and monitoring operation is performed by the actuating-and-sensing module 1. The gas transportation actuator 11 of the actuating-and-sensing module 1 is enabled to guide a specific amount of the gas to the gas sensor 12 and allows the gas sensor 12 to measure the gas. After the specific amount of the gas is transported from the gas transportation actuator 11 to the gas sensor 12, the gas sensor 12 acquires VOC concentration values of the gas during each monitoring time interval. The VOC concentration values are served as monitored values to be analyzed. In this embodiment, the monitoring time interval can be measured in seconds, for example each monitoring time interval may be 5 seconds.

In this embodiment, in a step S106, a calculating operation is performed. A plurality of the monitoring time intervals are referred as a time unit. During the time unit, the monitored values from the gas sensor 12 are added up so as to obtain a metabolism comparison value. The metabolism comparison value is served as a benchmark for determination.

Then, in a step S108, a comparing and warning operation is performed. It is noted that a warning threshold value is provided herein. The warning threshold value defines a critical amount that a human subject inhales per time unit. For example, a measured value that exceeds the warning threshold value may indicate that the concentration of the VOCs has adverse effects on the human subject. Therefore, the metabolism comparison value is compared with the warning threshold value. If the metabolism comparison value is larger than the warning threshold value, the actuating-and-sensing module 1 issues an emergency call, thereby providing the user with a protective measure. For example, the metabolism comparison value is obtained by summing up all the monitored values sensed by the gas sensor 12 during the time unit.

Please refer to Table 1, which is a total VOC guidelines issued by the German Federal Environmental Agency. If the concentration of total VOC is lower than 65 ppb (nL/L), the air quality level is excellent. If the concentration of total VOC is in the range between 65 ppb and 220 ppb, the air quality level is good and air circulation and ventilation in the environment is recommended. If the concentration of total VOC is in the range between 220 ppb and 660 ppb, the air quality level is moderate. In case that the air quality is moderate, intensified air circulation and ventilation in the environment is recommended, the sources of pollutants need to be searched and the exposure limit is smaller than 12 months. If the concentration of total VOC is in the range between 660 ppb and 2200 ppb, the air quality level is poor. In case that the air quality is poor, intensified air circulation and ventilation in the environment is necessary, the sources of pollutants need to be searched and the exposure limit is smaller than 1 month. If the concentration of total VOC is in the range between 2200 ppb and 5500 ppb, the air quality level is not healthy and the situation is unacceptable. In case that the air quality is not healthy and the situation is unacceptable, the air in the environment is used only if unavoidable, intense ventilation is necessary and the exposure limit is smaller than 1 hour. Ppb is a unit of measurement and is represented as nL/L. nL is equal to 10⁻⁹ liters.

TABLE 1
Total VOC guidelines issued by the German Federal
Environmental Agency

| | Hygienic | | Exposure | VOCs |
|---|---|---|---|---|
| Level | Rating | Recommendation | Limit | (ppb) |
| 5 | Situation not | Use only if unavoidable/Intense | hours | 2200-5500 |
| Unhealthy | acceptable | ventilation necessary | | |
| 4 | Major | Intensified ventilation | <1 month | 660-2200 |
| Poor | objections | necessary/Search for sources | | |
| 3 | Some | Intensified ventilation | <12 | 220-660 |
| Moderate | objections | recommended/Search for sources | months | |
| 2 | No relevant | Ventilation recommended | no limit | 65-220 |
| Good | objections | | | |
| 1 | No | Target value | no limit | 0-65 |
| Excellent | objections | | | |

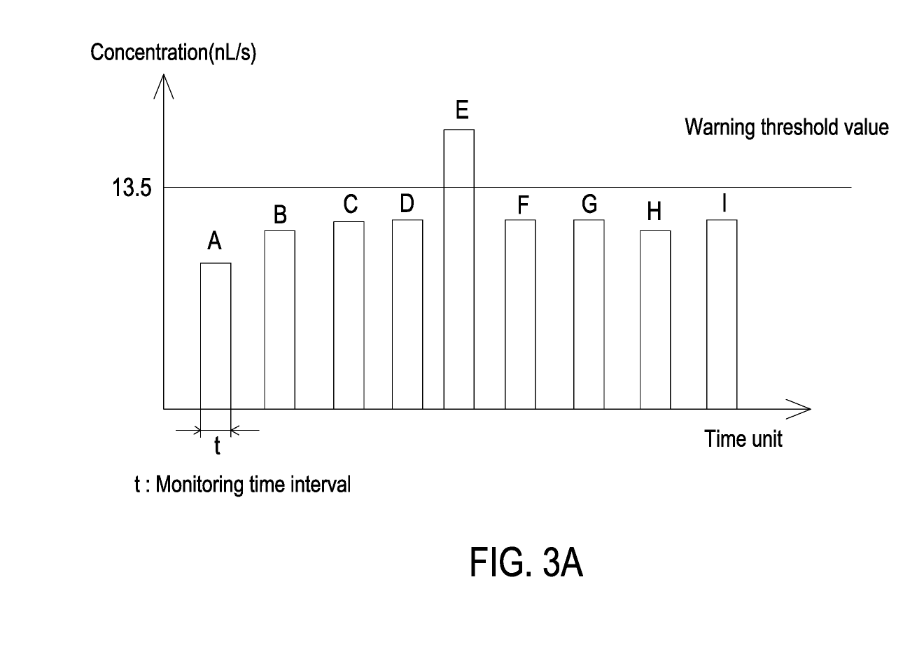

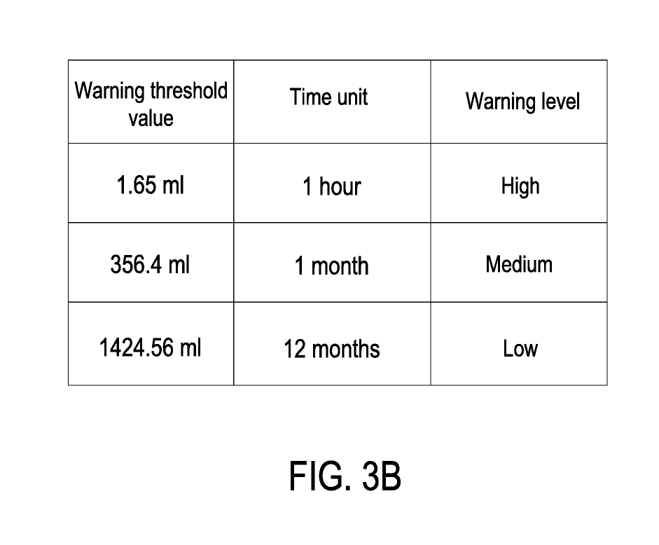

Hereinafter, some examples will be described according to the data of Table 1. FIG. 3A schematically illustrates a monitoring result of a gas sensor of the actuating-and-sensing module within a specific time unit according to the embodiment of the present disclosure. FIG. 3B schematically illustrates the relationships between warning threshold values and time units. Please refer to FIG. 3A and Table 1. If the concentration of total VOC is lower than 65 ppb (nL/L), the air quality level is excellent and no action should be taken by the user in the environment. In other words, 65 ppb is an upper limit of a safe exposure concentration. For example, the weight of the human body is 70 kilograms, and the air inhaled is 18,000 liters per day. The amount of the air inhaled per hour is 750 liters, and the total inhalation of VOCs is 750 L/h×65 ppb=13.5 nL/s. In other words, 13.5 nL per second is the upper limit of the safe exposure concentration, which may be severed as a warning threshold value. After the gas sensor 12 monitors the VOCs in each monitoring time interval t to generate each monitored value correspondingly, the microprocessor 13 receives the monitored values and calculates the monitored values subsequently. The microprocessor 13 compares the monitored values with the warning threshold value. If the monitored value is larger than the warning threshold value, the actuating-and-sensing module 1 issues the emergency call under control of the microprocessor 13. As shown in FIG. 3A, if the microprocessor 13 judges that the monitored value of the VOCs sensed by the gas sensor 12 during the monitoring time interval (e.g., the monitoring time intervals A, B, C, D, F, G H and I) is not larger than 13.5 nL/s, the actuating-and-sensing module 1 is disable to issue the emergency call under control of the microprocessor 13. On the contrary, if the microprocessor 13 judges that the monitored value of the VOCs sensed by the gas sensor 12 during the monitoring time interval (e.g., the monitoring time interval E) is larger than 13.5 nL/s, the actuating-and-sensing module 1 is enabled to issue the emergency call under control of the microprocessor 13.

As shown in FIG. 3A, the metabolism comparison value is obtained by summing up the monitored values of the VOCs per time unit (e.g., the monitoring time intervals A, B, C, . . . and I). Please refer to FIG. 3B and Table 1. The warning threshold values and the time units are acquired according to the data of Table 1, and the warning threshold values includes a high warning threshold value, a medium warning threshold value and a low warning threshold value. If the concentration of total VOC is larger than 2200 ppb and the exposure limit is smaller than 1 hour, the air quality level is not healthy. The high warning threshold value is determined according to the VOC inhalation amount of a human body corresponding to the VOC concentration of 2200 ppb within the time unit of 1 hour. For example, the weight of the human body is 70 kilograms, and the air inhalation is 18,000 liters per day. The amount of the air inhalation per hour is 750 liters, and the total inhalation of VOCs is 750 L/h×2200 ppb=458.3 nL/s. The VOC inhalation amount of the human body within the time unit of 1 hour is 458.3 nL/s×3600 sec=1.65 ml. In other words, 1.65 ml is the high warning threshold value corresponding to the time unit of 1 hour. If the metabolism comparison value obtained by summing up all of the monitored values, which are sensed by the gas sensor 12 and corresponding to the monitoring time intervals within the time unit of 1 hour, is larger than the high warning threshold value (e.g., 1.65 ml), the actuating-and-sensing module 1 issues the emergency call in real time to provide the protection measure to the user. For example, the user may leave the environment rapidly. The air in the environment is used only if unavoidable, and intense air circulation and ventilation is necessary.

If the concentration of total VOC is larger than 660 ppb and the exposure limit is smaller than 1 month, the air quality level is poor. The medium warning threshold value is determined according to the VOC inhalation amount of the human body corresponding to the VOC concentration of 660 ppb within the time unit of 1 month. For example, the weight of the human body is 70 kilograms, and the air inhalation is 18,000 liters per day. The amount of the air inhalation per hour is 750 liters, and the total inhalation of VOCs is 750 L/h×660 ppb=137.5 nL/s. The VOC inhalation amount of the human body within the time unit of 1 month is 137.5 nL/s×3600 sec×24 h×30=356.4 ml. In other words, 356.4 ml is the medium warning threshold value corresponding to the time unit of 1 month. If the metabolism comparison value obtained by summing up all of the monitored values, which are sensed by the gas sensor 12 and corresponding to the plural monitoring time intervals within the time unit of 1 month, is larger than the medium warning threshold value (e.g., 356.4 ml), the actuating-and-sensing module 1 issues the emergency call in real time to provide the protective measure to the user. For example, the sources of pollutants need to be searched, the air in the environment is used only if unavoidable, and intensified air circulation and ventilation is necessary.

If the concentration of total VOC is larger than 220 ppb and the exposure limit is smaller than 12 months, the air quality level is moderate. The low warning threshold value is determined according to the VOC inhalation amount of the human body corresponding to the VOC concentration of 220 ppb within the time unit of 12 months. For example, the weight of the human body is 70 kilograms, and the air inhalation is 18,000 liters per day. The amount of the air inhalation per hour is 750 liters, and the total inhalation of VOCs is 750 L/h×220 ppb=45.8 nL/s. The VOC inhalation amount of the human body within the time unit of 12 months is 45.8 nL/s×3600 sec×24 h×30×12=1424.56 ml. In other words, 1424.56 ml is the low warning threshold value corresponding to the time unit of 12 months. If the metabolism comparison value obtained by summing up all of the monitored values, which are sensed by the gas sensor 12 and corresponding to the plural monitoring time intervals within the time unit of 12 months, is larger than the low warning threshold value (e.g., 1424.56 ml), the actuating-and-sensing module 1 issues the emergency call in real time to provide the protective measure to the user. For example, intensified air circulation and ventilation is recommended and the sources of pollutants need to be searched.

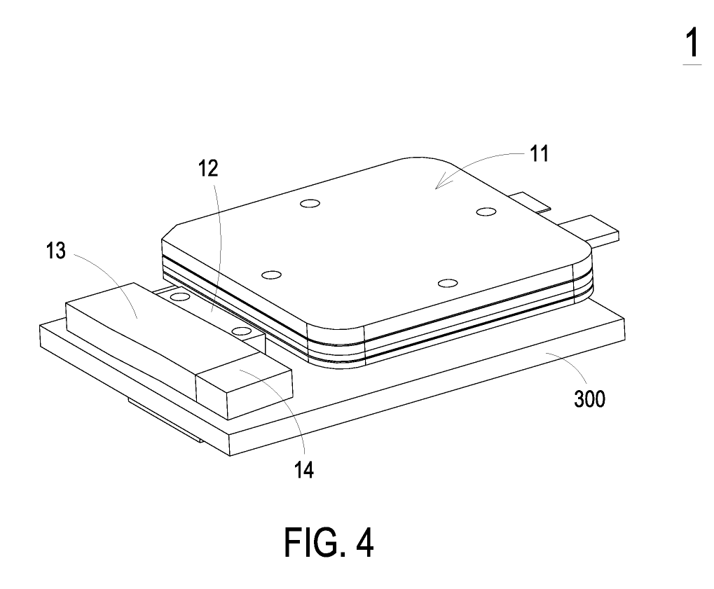

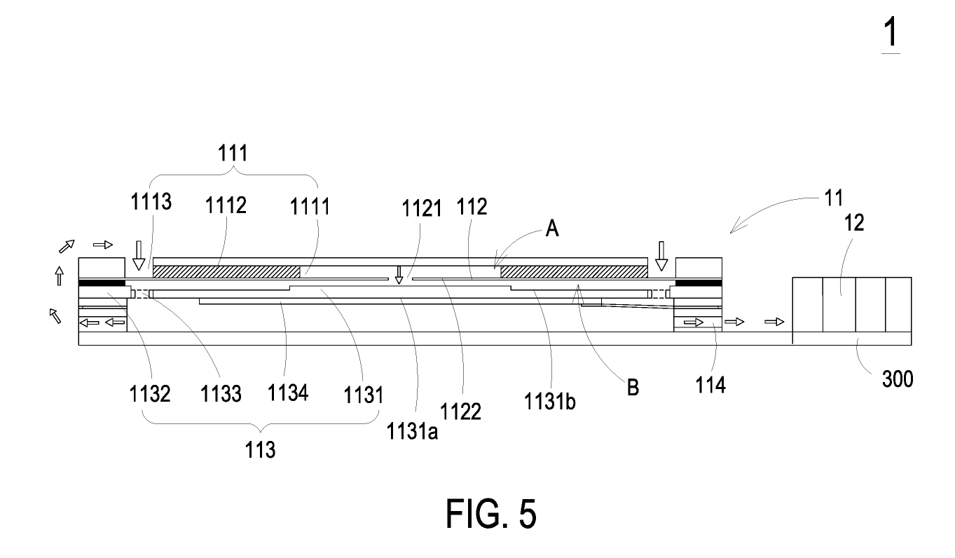

Please refer to FIGS. 4 and 5. FIG. 4 is a schematic perspective view illustrating the actuating-and-sensing module according to the embodiment of the present disclosure. FIG. 5 is a schematic cross-sectional view illustrating the actions of the actuating-and-sensing module according to the embodiment of the present disclosure. The gas transportation actuator 11, the gas sensor 12, the microprocessor 13 and the transmission module 14 of the actuating-and-sensing module 1 are disposed on a carrier 300. The gas transportation actuator 11 includes a gas inlet plate 111, a resonance plate 112 and a piezoelectric actuator 113. The gas inlet plate 111 includes a central cavity 1111, at least one convergence channel 1112 and at least one inlet 1113. The central cavity 1111 forms a convergence chamber A. After the gas is inhaled through the inlet 1113, the gas is guided through the convergence channel 1112, which is spatially corresponding to the inlet 1113, to the convergence chamber A. The resonance plate 112 has a central aperture 1121 spatially corresponding to the convergence chamber A. Moreover, the resonance plate 112 has a movable part 1122 surrounding the central aperture 1121.

The piezoelectric actuator 113 is corresponding in position to the resonance plate 112. The piezoelectric actuator 113 includes a suspension plate 1131, an outer frame 1132, at least one bracket 1133 and a piezoelectric plate 1134. The suspension plate 1131 has a first surface 1131a and a second surface 1131b. The suspension plate 1131 can be subjected to a bending vibration. The outer frame 1132 is arranged around the suspension plate 1131. The at least one bracket 1133 is connected between the suspension plate 1131 and the outer frame 1131, so that the bracket 1133 can elastically support the suspension plate 1131. The length of a side of the piezoelectric plate 1134 is smaller than or equal to the length of a side of the suspension plate 1131. Moreover, the piezoelectric plate 1134 is attached on the first surface 1131a of the suspension plate 1131 and is subjected to a deformation in response to an applied voltage so as to drive the suspension plate 1131 to undergo the bending vibration. There is a gap between the resonance plate 112 and the piezoelectric actuator 113 so as to define a first chamber B.

When the piezoelectric actuator 113 of the gas transportation actuator 11 is enabled, the piezoelectric plate 1134 is subjected to deformation so as to drive the suspension plate 1131 to vibrate in a reciprocating manner by using the bracket 1133 as a fulcrum. The piezoelectric actuator 113 is in resonance with the movable part 1122 of the resonance plate 112, so that the first chamber B is vibrated to generate a pressure gradient for inhaling the ambient gas into the inlet 1113 of the gas inlet plate 111 to form a gas flow. Then, the gas is transported to the convergence chamber A of the central cavity 1111 through the at least one convergence channel 1112. Then, the gas is transported to the first chamber B through the central aperture 1121 of the resonance plate 112. Then, the gas is transported downwardly to the region between the piezoelectric actuator 113 and the carrier 300 through the vacant space between the brackets 1133. Finally, the gas is ejected to the gas sensor 12 through an outlet channel 114. Since the gas is transported from the gas transportation actuator 11 to the gas sensor 12 at a specific transportation amount, the gas sensor 12 can monitor the gas in consistent with the external environment so as to acquire the accurate detection result in real time.

From the above descriptions, the present disclosure provides a VOC detecting and warning method, which utilizes the gas transportation actuator of the actuating-and-sensing module to guide a specific amount of the gas to the gas sensor for monitoring the VOCs of the gas. Consequently, the accuracy of the VOC detection is increased. When the monitored value of the VOCs corresponding to the monitoring time interval is larger than an upper limit of a safe exposure concentration, the actuating-and-sensing module issues an emergency call to warn the user that the VOC concentration value in the environment is high. In addition, all of the monitored values of the VOCs, which are sensed by the gas sensor and corresponding to the monitoring time intervals per time unit, are added up to obtain a metabolism comparison value. If the metabolism comparison value is larger than a warning threshold value, the actuating-and-sensing module issues an emergency call to provide a protective measure to the user. Consequently, the cumulative damage caused by the exposure to VOCs in the located environment can be avoided.

While the disclosure has been described in terms of what is presently considered to be the most practical and preferred embodiments, it is to be understood that the disclosure needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A VOC (volatile organic compound) detecting and warning method, comprising steps of:
(a) providing an actuating-and-sensing module, wherein the actuating-and-sensing module comprises a gas transportation actuator and a gas sensor;
(b) performing a gas-guiding and monitoring operation, wherein the gas transportation actuator is enabled to guide a specific amount of gas to the gas sensor, and the gas sensor monitors a volatile organic compound of the specific amount of the gas in each monitoring time interval and obtains each monitored value corresponding thereto;
(c) performing a calculating operation, wherein a plurality of the monitoring time intervals are a time unit, and the monitored values during the time unit are added up to obtain a metabolism comparison value; and
(d) performing a comparing and warning operation, wherein an upper limit of the VOC that a human subject inhales per time unit defines a warning threshold value, and the metabolism comparison value is compared with the warning threshold value, wherein if the metabolism comparison value is larger than the warning threshold value, the actuating-and-sensing module issues an emergency call, thereby providing a user with a protective measure.

2. The VOC detecting and warning method according to claim 1, wherein the VOC detecting and warning method in the step (b) further comprises a step (1):
allowing the actuating-and-sensing module to issue the emergency call if the monitored value is larger than a warning threshold value during the monitoring time interval.

3. The VOC detecting and warning method according to claim 1, wherein the actuating-and-sensing module further comprises a microprocessor and a transmission module, wherein the microprocessor processes and converts information of the monitored values into an output data and controls the actuation of the gas transportation actuator, wherein the transmission module transmits the output data, which is processed and converted by the microprocessor, to a connection device, so that the connection device displays, stores and transmits information of the output data.

4. The VOC detecting and warning method according to claim 3, wherein the connection device performs a protective measure for notification.

5. The VOC detecting and warning method according to claim 3, wherein the connection device is a display device having a wired communication module, and the display device is used to perform a protective measure that displays an image for notification.

6. The VOC detecting and warning method according to claim 3, wherein the connection device is a display device having a wireless communication module, and the display device is used to perform a protective measure that displays an image for notification.

7. The VOC detecting and warning method according to claim 3, wherein the connection device is a portable electronic device having a wireless communication module, and the portable electronic device is used to perform a protective measure for notification, wherein the protective measure is at least one selected from the group consisting of displaying an image, generating a sound effect, generating a light effect and generating a vibration effect.

8. The VOC detecting and warning method according to claim 5, wherein the wired transmission module is at least one selected from the group consisting of a USB transmission module, a mini-USB transmission module and a micro-USB transmission module.

9. The VOC detecting and warning method according to claim 6, wherein the wireless transmission module is at least one selected from the group consisting of a Wi-Fi transmission module, a Bluetooth transmission module, a radio frequency identification transmission module and a near field communication transmission module.

10. The VOC detecting and warning method according to claim 1, wherein the gas transportation actuator comprises:
a gas inlet plate having at least one inlet, at least one convergence channel and a central cavity defining a convergence chamber, wherein the at least one inlet allows the gas to flow in, and wherein the convergence channel is spatially corresponding to the inlet and guides the gas flowing in the inlet to the convergence chamber;
a resonance plate having a central aperture and a movable part, wherein the central aperture is spatially corresponding to the convergence chamber and the movable part surrounds the central aperture; and
a piezoelectric actuator aligned with the resonance plate,
wherein a gap is formed between the resonance plate and the piezoelectric actuator to define a first chamber, so that the gas flowing in the at least one inlet of the gas inlet plate is converged to the central cavity along the at least one convergence channel and flows into the first chamber through the central aperture of the resonance plate when the piezoelectric actuator is enabled, whereby the gas is further transported through a resonance between the piezoelectric actuator and the movable part of the resonance plate.

11. The VOC detecting and warning method according to claim 10, wherein the piezoelectric actuator comprises:
a suspension plate having a first surface and a second surface, wherein the suspension plate is permitted to undergo a bending vibration;
an outer frame arranged around the suspension plate;
at least one bracket connected between the suspension plate and the outer frame for elastically supporting the suspension plate; and
a piezoelectric plate, wherein a length of a side of the piezoelectric plate is smaller than or equal to a length of a side of the suspension plate, and the piezoelectric plate is attached on the first surface of the suspension plate, wherein when a voltage is applied to the piezoelectric plate, the suspension plate is driven to undergo the bending vibration.

12. A VOC (volatile organic compound) detecting and warning method, comprising steps of:
(a) providing at least one actuating-and-sensing module, wherein the actuating-and-sensing module comprises at least one gas transportation actuator and at least one gas sensor;
(b) performing a gas-guiding and monitoring operation, wherein the gas transportation actuator is enabled to guide at least one specific amount of gas to the gas sensor, and the gas sensor monitors at least one volatile organic compound of the specific amount of the gas in each monitoring time interval and obtains at least one monitored value corresponding thereto;
(c) performing a calculating operation, wherein a plurality of the monitoring time intervals are a time unit, and the monitored values are added up during the time unit to obtain at least one metabolism comparison value; and
(d) performing a comparing and warning operation, wherein at least one upper limit of the VOC that a human subject inhales per time unit defines at least one warning threshold value, and the metabolism comparison value is compared with the warning threshold value, wherein if the metabolism comparison value is larger than the warning threshold value, the actuating-and-sensing module issues at least one emergency call, thereby providing at least one protective measure to a user.
